(19) United States
(12) Patent Application Publication
(10) Pub. No.: US 2006/0079698 A1
(43) Pub. Date: Apr. 13, 2006

(54) Process for the preparation of intermediates of trandolapril and use thereof for the preparation of trandolapril

(75) Inventors: Narendra Shriram Joshi, Navi Mumbai (IN); Shekhar Bhaskar Bhirud, Navi Mumbai (IN); Buddhavarapu Pattabhi Ramam, Navi Mumbai (IN); Arjun Rajaram Bodkhe, Thane (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/245,871

(22) Filed: Oct. 7, 2005

Related U.S. Application Data
(60) Provisional application No. 60/616,934, filed on Oct. 7, 2004.
(60) Provisional application No. 60/616,959, filed on Oct. 7, 2004.

(51) Int. Cl. C07D 209/42

(52) U.S. Cl. 548/492

(57) ABSTRACT

A process for the preparation of an intermediate of trandolapril, (2S,3aR,7aS)-perhydroindole-2-carboxylic acid of Formula II 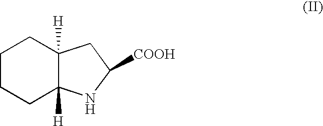 is provided. Also provided are processes for preparing trandolapril.

This application claims the benefit under 35 U.S.C. §119 to Provisional Application No. 60/616,934, filed Oct. 7, 2004 and entitled “PROCESS FOR THE PREPARATION OF INTERMEDIATES OF TRANDOLAPRIL”, and from Provisional Application No. 60/616,959, filed Oct. 7, 2004 and entitled “PROCESS FOR THE PREPARATION OF TRANDOLAPRIL”, the contents of each of which are incorporated by reference herein.

### BACKGROUND OF THE INVENTION

1. Technical Field

The present invention generally relates to an improved process for the preparation of intermediates for trandolapril and to processes for the preparation of trandolapril.

2. Description of the Related Art

Trandolapril, also known as (2S,3aR,7aS)-1-[(S)-N-[(S)-1-carboxy-3-phenylpropyl]alanyl]hexahydro-2-indolinecarboxylic acid, 1-ethyl ester, is represented by the structure of Formula I. 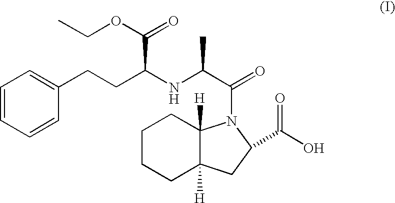
Generally, trandolapril deesterifies to the diacid metabolite, trandolaprilat, which is an active inhibitor of angiotensin-converting enzyme (“ACE”) activity. ACE is a peptidyl dipeptidase that catalyzes the conversion of angiotensin I to the vasoconstrictor, angiotensin II. Angiotensin II is a potent peripheral vasoconstrictor that also stimulates secretion of aldosterone by the adrenal cortex and provides negative feedback for renin secretion. The effect of trandolapril in hypertension appears to result primarily from the inhibition of circulating and tissue ACE activity thereby reducing angiotensin II formation, decreasing vasoconstriction, decreasing aldosterone secretion, and increasing plasma renin. Trandolapril is indicated for use in treating hypertension. Trandolapril is sold under the trade name MAVIK®.

(2S,3aR,7aS)-perhydroindole-2-carboxylic acid is a key intermediate in the formation of trandolapril. For example, U.S. Pat. No. 4,933,361 discloses a process for the preparation of trandolapril comprising the reaction between N-[(S)-1-(ethoxycarbonyl)-3-phenylpropyl]-L-alanine and either a benzyl or tert-butyl protected (2S,3aR,7aS)-perhydroindole-2-carboxylic acid using a condensing agent such as dicyclohexylcarbodiimide or 1-hydroxybenzotriazole followed by hydrolysis of the protection group.

### SUMMARY OF THE INVENTION

In accordance with one embodiment of the present invention, a process for the preparation of an intermediate of trandolapril, (2S,3aR,7aS)-perhydroindole-2-carboxylic acid of Formula II: 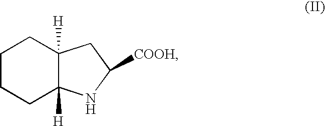
is provided, the process comprising:
(a) esterifying (3aR,7aS)-perhydroindole-2-carboxylic acid of Formula III: 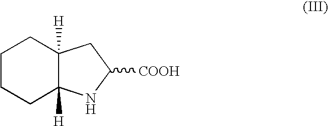
   with an alcohol of the formula ROH wherein R is an aliphatic, aryl or aralkyl group and a suitable acid to provide an acid additional salt of a compound of Formula IV: 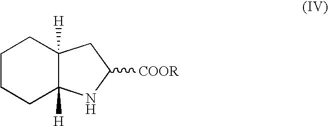
   wherein R has the aforestated meanings;
(b) reacting the acid addition salt compound of Formula IV with a first base to provide a compound of Formula V: 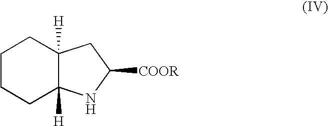
(c) reacting the compound of Formula V with dibenzoyl-L-tartaric acid or di-p-toluoyl-L-tartaric acid and at least one lower aliphatic alcohol of the formula R¹OH wherein R¹ is an alkyl group containing from 1 to about 12 carbon atoms, and adding a second base to precipitate a compound of Formula VI: 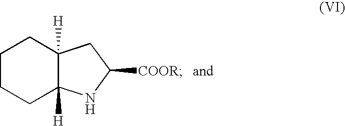
(d) hydrolyzing the compound of Formula VI to provide the compound of Formula II.

The advantages of this process of the present invention includes at least:

1. The use of (3aR,7aS)-perhydroindole-2-carboxylic acid as a starting material, which is readily available, cost effective and does not require arduous separation of carboxyperhydroindole isomers.

2. The process for preparing does not require the use of expensive catalysts for debenzylation.

3. The process provides relatively high yields with a relatively short process.

In accordance with a second embodiment of the present invention, a process for the preparation of trandolapril is provided comprising: (a) reacting N-[(S)-1-(ethoxycarbonyl)-3-phenylpropyl]-L-alanine and N,N′-carbonyldiimidazole in the presence of a solvent to form an N-carboxyanhydride (NCA); and (b) reacting the NCA with (2S,3aR,7aS)-perhydroindole-2-carboxylic acid to provide trandolapril.

The advantages of this process of the present invention includes at least:

1. The reaction to form N-carboxyanhydride provides an economical and effective industrial process for the preparation of trandolapril because the condensation reaction may be performed at relatively lower temperatures more favorable to industrial scale processes.

2. The formation of N-carboxyanhydride is economical because the solvents used may be recycled.

3. The (2S,3aR,7aS)-perhydroindole-2-carboxylic acid may be used without protecting the carboxylic acid group.

### DETAILED DESCRIPTION OF THE PREFERRED EMBODIMENTS

One embodiment of the present invention is a process for producing an intermediate of trandolapril, (2S,3aR,7aS)-perhydroindole-2-carboxylic acid of Formula II: 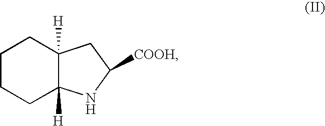
In step (a) of the process of the present invention, (3aR,7aS)-perhydroindole-2-carboxylic acid of Formula III 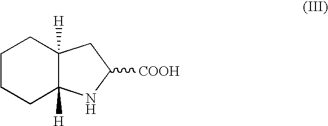
is esterfied with an alcohol of the formula ROH wherein R is an aliphatic, aryl or aralkyl group and a suitable acid to provide an acid addition salt of a compound of Formula IV under esterification conditions known in the art. 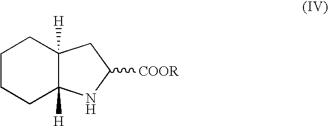
wherein R has the aforestated meanings. Suitable alcohols include, but are not limited to, linear or branched C₁-C₃₀ aliphatic alcohols, C₆-C₃₀ aryl and araalkyl alcohols and the like and mixtures thereof. Examples of useful alcohols include, but are not limited to, methyl alcohol, ethyl alcohol, propyl alcohol, isopropyl alcohol, butyl alcohol, benzyl alcohol and the like and mixtures thereof with benzyl alcohol being preferred for use herein. Suitable acids for forming the acid addition salt compound of Formula IV include, but are not limited to, sulfonic acids such as p-toluenesulfonic acid, hydrochloric acid (e.g., hydrochloric acid gas) and the like and mixtures thereof.

The esterification reaction is advantageously carried out in a solvent. Useful solvents include, but are not limited to, aromatic hydrocarbons, aliphatic hydrocarbons, halogenated aliphatic hydrocarbons, ethers, nitriles and the like and mixtures thereof. Suitable substituted or unsubstituted aromatic hydrocarbons include C₆-C₁₄ aromatic hydrocarbons such as, for example, benzene, toluene, o-, m-, and p-xylene (single or mixed isomers), ethylbenzene and the like and mixtures thereof. Suitable ethers include dialkyl ethers wherein each alkyl is independently a C₁-C₆ alkyl, linear and branched C₁-C₆ alkanes substituted with two O—C₁-C₆ alkyl groups (which are the same or different), C₄-C₈ cyclic ethers and diethers, C₆-C₈ aromatic ethers such as, for example, tetrahydrofuran, diethylether, tert-butylmethylether, diisopropylether, di-n-butyl ether, dioxane, and the like and mixtures thereof. Suitable nitriles include C₂-C₆ aliphatic nitriles such as, for example, acetonitrile, propionitrile and the like and mixtures thereof. Suitable halogenated aliphatic hydrocarbons include, but are not limited to, carbon tetrachloride, chloroform, methylene chloride, 1,2-dichloroethane, 1,1,2-trichloroethane, 1,1,2,2-tetrachloroethane and the like and mixtures thereof. The solvent can be present in an amount ranging from about 10 to about 15 volumes.

The esterification reaction can be performed by heating the solution to reflux, e.g., a temperature ranging from about 50° C. to about 125° C. and preferably from about 80° C. to about 115° C. The time period for the reaction can range from about 3 hours to about 7 hours. The alcohol will ordinarily be present in the reaction in an amount ranging from about 1.5 molar equivalent to about 2 molar equivalent per equivalent of the compound of Formula III while the acid will ordinarily be present in an amount ranging from about 1 molar equivalent to about 1.5 molar equivalent per molar equivalent per equivalent of the compound of Formula III.

In step (b) of the process of the present invention, the compound of Formula IV is reacted with a first base to provide a compound of Formula V.

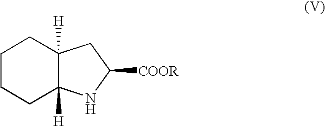

In one embodiment, the base of step (b) is an amine. Suitable amines include, but are not limited to, primary amines, secondary amines, tertiary amines, aliphatic amines, aromatic amines and the like and mixtures thereof. In one embodiment, the amine base is one or more tertiary amines. In another embodiment, the amine base is one or more trialkylamines. Useful tertiary amine base groups include, but are not limited to, tri(lower alkyl)amines containing from about 4 to about 20 carbon atoms such as trimethylamine, triethylamine, tripropylamine, tributylamine and the like and mixtures thereof. Preferred amines for use herein are triethylamine and ammonia and mixtures thereof. In another embodiment, the base of step (b) includes, but is not limited to, alkali metal carbonates such as sodium carbonate and potassium carbonate and the like and mixtures thereof. Generally, the base is present in the range of about 1 molar equivalent to about 2.5 molar equivalents per equivalent of the compound of Formula IV.

In step (c) of the process of the present invention, the compound of Formula V is reacted with dibenzoyl-L-tartaric acid or di-p-toluoyl-L-tartaric acid and at least one lower aliphatic alcohol of the formula R¹OH wherein R¹ is an alkyl group containing from 1 to about 12 carbon atoms and preferably from 1 to 6 carbon atoms. Suitable alcohols include, but are not limited to, methanol, ethanol, butanol, propanol, isopropyl alcohol and the like and mixtures thereof. Generally, the reaction can be performed at a temperature ranging from about 20° C. to about 80° C. from about 25° C. to about 35° C. The time period for completing the reaction will ordinarily range from about 2 hours to about 4 hours. The molar ratio of the compound of Formula V to dibenzoyl-L-tartaric acid or di-p-toluoyl-L-tartaric acid will generally range from about 1:0.9 to about 1:1.2.

Following the reaction of the compound of Formula V with dibenzoyl-L-tartaric acid and a lower alcohol, a second base is added to the reaction mixture to precipitate out a compound of Formula VI. 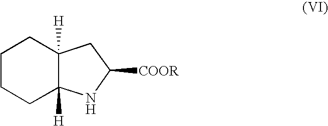
The second base includes, but is not limited to, alkali metal hydroxides, and the like and mixtures thereof. Useful alkali metal hydroxides include, but are not limited to, sodium hydroxide, potassium hydroxide and the like and mixtures thereof.

In step (d) of the process of the present invention, the compound of Formula VI is hydrolyzed with a hydrolizing agent such as HCl in the presence of a suitable base, such as an alkali metal hydroxide, e.g., sodium hydroxide, and in the presence of a suitable solvent, such as an alcohol, e.g. methanol and the like to provide the compound of Formula II. If desired, the compound of Formula II can be purified and/or converted to trandolapril, e.g., by reacting the compound of Formula II with N,N′-carbonyldiimidazole in the presence of a solvent to form an N-carboxyanhydride (NCA); and reacting the NCA with (2S,3aR,7aS)-perhydroindole-2-carboxylic acid to provide trandolapril as described herein below.

Another embodiment of the present invention involves a process for producing trandolapril. Generally, the process includes at least (a) reacting N-[(S)-1-(ethoxycarbonyl)-3-phenylpropyl]-L-alanine (VII) and N,N′-carbonyldiimidazole (CDI, VIII) in the presence of a solvent to form an N-carboxyanhydride (NCA, IX); and (b) reacting the NCA (IX) with (2S,3aR,7aS)-perhydroindole-2-carboxylic acid (II) to provide trandolapril (I) as generally shown in Scheme I:

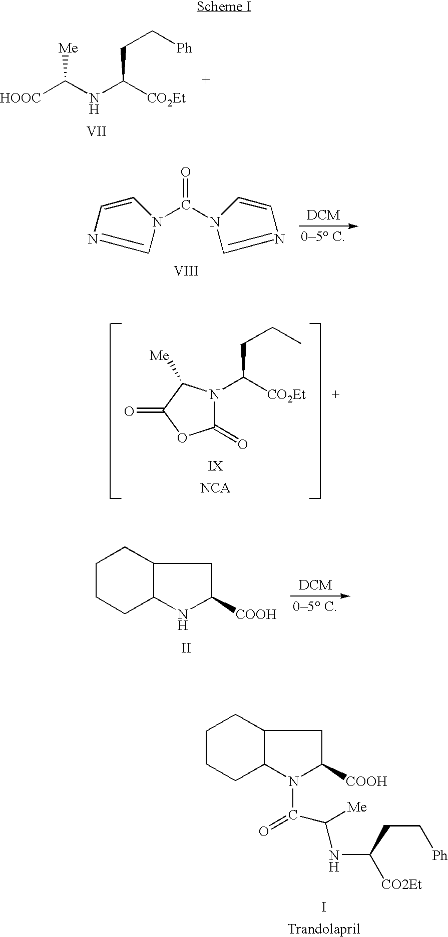

Useful solvents in the process of the present invention include, but are not limited to, one or more aprotic solvents and the like. Suitable aprotic solvents include, but are not limited to, halogenated hydrocarbons, e.g., dichloromethane, dichloroethane, etc.; heterocyclic compounds, e.g., tetrahydrofuran, amides, e.g., dimethylformamide, and the like and mixtures thereof. Preferred solvents for use herein are dichloromethane, dichloroethane, tetrahydrofuran, dimethylformamide and mixtures thereof.

The reaction between the NCA (VIII) and (2S,3aR,7aS)-perhydroindole-2-carboxylic acid (II) may be performed in situ without isolation of the NCA. When the reaction is performed in this manner, it takes place in the same solvent as the reaction in the first step. The reaction may be performed at a temperature ranging from about −10° C. to about 15° C., and preferably at a temperature ranging from about 0° C. to about 5° C. The reaction time ranges from about 1 hour to about 4 hours, and preferably from about 2 hours to about 3 hours.

The trandolapril thus obtained may be purified and isolated using suitable solvent such as, for example, isopropyl alcohol, acetone, ethyl acetate, toluene or acetonitrile by conventional techniques.

In another embodiment of the present invention, N-[(S)-1-(ethoxycarbonyl)-3-phenylpropyl]-L-alanyl halide (X) is formed by reaction of N-[(S)-1-(ethoxycarbonyl)-3-phenylpropyl]-L-alanine (VII) with a halogenating agent known in the art in the presence or absence of an inert gas. The N-[(S)-1-(ethoxycarbonyl)-3-phenylpropyl]-L-alanyl halide of formula (X) is then reacted with (2S,3aR,7aS)-perhydroindole-2-carboxylic acid (II) in an organic solvent at low to ambient temperature and in the presence of a base to provide trandolapril (I) as generally shown in Scheme II. 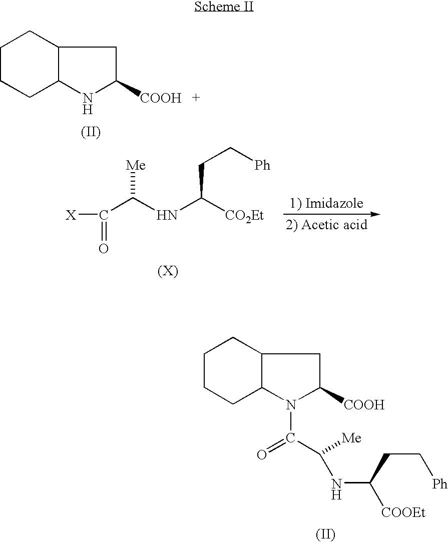
wherein X is a halide such as chloride and Ph is phenyl.

The reaction of the compound of Formula X with a compound of Formula II can advantageously be conducted in one or more organic solvents, and preferably an anhydrous solvent. Examples of suitable organic solvents include chlorinated hydrocarbons, e.g., dichloromethane and dichloroethane; aromatic hydrocarbons, e.g., benzene and toluene; aliphatic hydrocarbons, e.g., hexane and heptane, cycloalkyl hydrocarbons, e.g., cyclopentane and cyclohexane, and the like and mixtures thereof. Preferably, the organic solvent is a chlorinated hydrocarbon. Suitable inert gases include, but are not limited to, nitrogen, argon, etc. The reaction can be carried out at low to ambient temperatures ranging from about −20° C. to about 30° C., and preferably from about 5° C. to about 10C. The time required for completion of reaction can range from about 1 hour to about 12 hours depending on the temperature employed. The molar proportions of the (2S,3aR,7aS)-perhydroindole-2-carboxylic acid (II) employed can be from about 0.9 moles to about 1.0 moles per mole of the compound of Formula X.

Additionally, the reaction can be carried out in presence of one or more organic bases such as, for example, dialkylamines, e.g., diethyl amine, trialkylamines, e.g., triethyl amine, heterocyclic amines, e.g., pyridines, imidazoles, n-methyl morpholine, and the like. A preferred base is imidazole. Typically, the base is employed in molar proportions of about 2.0 to about 5.0 moles per mole of compound of the Formula X, and preferably from about 2.0 to about 4.0 moles.

After completion of the reaction, the pH may be adjusted to about 4 to about 5 by addition of a suitable mineral acid. The organic layer may then be separated by known techniques, washed and dried. The solvents can then be substantially evaporated to obtain a solid. If desired, the solid trandolapril of Formula I prepared by the process of the present invention can be purified by crystallization by conventional techniques, e.g., such as from ethanol/water or ethyl acetate/hexane. The process of the present invention advantageously provides trandolapril of Formula I in relatively high purity, e.g., greater than about 97%, preferably greater than about 98% and more preferably greater than about 99%.

In another embodiment of the present invention, (2S,3aR,7aS)-perhydroindole-2-carboxylic acid (II) can be converted to a silyl ester of Formula XI by reacting the compound of Formula II with a trialkyl or aryl silyl halide, e.g., chloride, in the presence of one or more organic base and in situ reacted with N-[(S)-1-(ethoxycarbonyl)-3-phenylpropyl]-L-alanyl halide (X) in an organic solvent at low to ambient temperature and in the presence of a base followed by treatment with a suitable acid, e.g., acetic acid, to obtain trandolapril of the formula (I) as generally set forth in Scheme III.

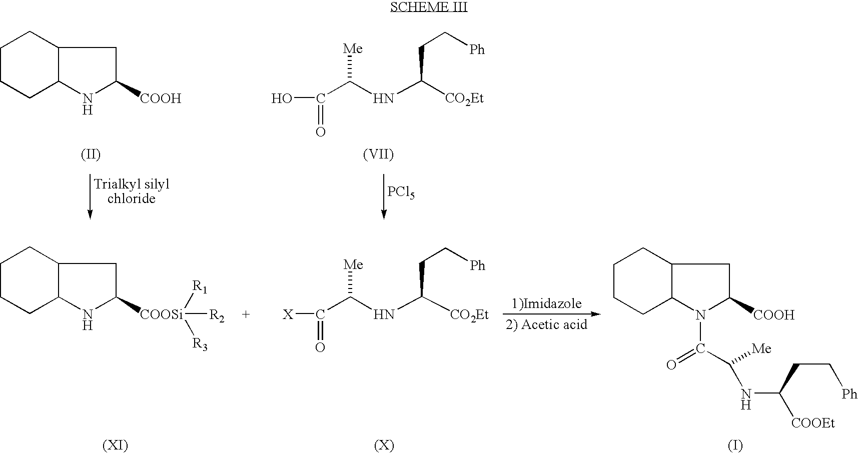

The trandolapril may be purified and isolated using a suitable solvent such as, for example, isopropyl alcohol, acetone, ethyl acetate, toluene or acetonitrile, by conventional techniques.

The following examples are provided to enable one skilled in the art to practice the invention and are merely illustrative of the invention. The examples should not be read as limiting the scope of the invention.

### EXAMPLE 1

### Preparation of (2S,3aR,7aS)-perhydroindole-2-carboxylic acid

A mixture of (3aR,7aS)-perhydroindole-2-carboxylic acid (150 g), toluene (1.5 L), p-toluenesulfonic acid (202.6 g) and benzyl alcohol (162.95 g) were mixed in a vessel. The mixture was heated until reflux and the water was removed by azeotropic distillation. After removal of the water, the reaction mixture was refluxed for an additional 3 hours. After completion of the reaction as determined by thin layer chromatography (TLC), the reaction mixture was slowly cooled to a temperature of about 25° C. The product was filtered off and washed with toluene (300 ml) to yield (3aR,7aS)-benzyl-perhydroindole-2-carboxylate PTSA salt (300 g).

Infra Red (IR) (KBr, cm-1): 2938, 1748, 1241, 1160, 1009, 681. The 1H-NMR (CDCl3): δ 1-2.3 (m, 14H), 2.92 (td, 1H), 4.15(d, 1H), 5-5.25 (m, 3H), 7.1-7.8 (m, 9H).

(3aR,7aS)-benzyl-perhydroindole-2-carboxylate PTSA salt (300 g) was charged with methylene dichloride (3 L). Triethylamine (193.9 ml) was added and the mixture was stirred for about 30 minutes. The organic phase was separated and washed with water twice (1.5 L×2). The organic layer was then concentrated to yield (2S,3aR,7aS)-benzyl-perhydroindole-2-carboxylate (180 g) as an oil.

The above obtained oil was added to a flask and methanol (0.9 L) and a solution of dibenzoyl-L-tartaric acid (249 g) in methanol (0.9 L) were added to the flask at a temperature of about 25° C. The reaction mixture was stirred for about 30 minutes and then heated to a temperature of about 60° C. for about 1 hour. The reaction mixture was cooled to a temperature of about 15° C. The solid was filtered off and washed with methanol (180 ml). The solid was dried at a temperature of about 60° C. to yield the tartarate salt of (2S,3aR,7aS)-benzyl-perhydroindole-2-carboxylate (180 g).

The (2S,3aR,7aS)-benzyl-perhydroindole-2-carboxylate tartarate salt (180 g) was added to methylene chloride (1.8 L) and charged with a solution of sodium hydroxide (23.4 g) in water (0.9 L). The reaction mixture was stirred for about 1 hour. The organic layer was separated and washed with water (900 ml) and then with a brine water solution (900 ml). The organic phase was separated and the methylene chloride was removed to yield (2S,3aR,7aS)-benzyl-perhydroindole-2-carboxylate (72 g).

(2S,3aR,7aS)-benzyl-perhydroindole-2-carboxylate (70 g) was added to methanol (350 ml) and charged with a solution of sodium hydroxide (13.4 g) in water (50 ml). The reaction mixture was refluxed for about 2 hours. After completion of reaction as determined by TLC, the pH of the reaction mixture was adjusted to a range of from about 6 to about 7 with dilute hydrochloric acid. The methanol was concentrated and charged with ethanol (700 ml). The reaction mixture was heated to a temperature of about 70° C. and the inorganics were filtered off. The ethanol was concentrated and the product was isolated with acetone (700 ml). The product was dried at a temperature of about 60° C. to yield (2S,3aR,7aS)-perhydroindole-2-carboxylic acid (42 g).

IR (Nujol, cm-1): 2923, 2854, 1600, 1458, 1377, 1319. 1H-NMR (D2O): δ 1.1-2.5 (m, 8H), 1.65(m,1H), 1.96-2.37 (m,2H), 2.91(td, 1H),4.46(d, 1H). Mass (m/z): 168.3(M-H).

### EXAMPLE 2

### Preparation of Trandolapril

N-[(S)-1-(ethoxycarbonyl)-3-phenylpropyl]-L-alanine (33 g) was added to a flask with methylene dichloride (500 ml) and cooled to a temperature of about 0° C. N,N′-carbonyl diimidazole (19.16 g) was added to the mixture at the same temperature. The reaction was maintained at a temperature ranging from about 0° C. to about 5° C. for about 1 hour. (2S,3aR,7aS)-perhydroindole-2-carboxylic acid (30 g) of Example 1 was added to the reaction mixture and the reaction temperature was maintained at a temperature ranging from about 0° C. to about 5° C. for an about 2 hours. Methylene dichloride was distilled off and acetic acid (4.19 g) in water (500 ml) was added to the reaction mixture. The aqueous layer was saturated with sodium chloride and the product was extracted continuously with methylene dichloride. Methylene dichloride was then distilled off. The residue was dissolved in isopropyl alcohol (165 ml) and the product was isolated to produce trandolapril (30 g).

IR (KBr, cm-1): 3444, 3280, 2973, 2942, 2881, 1735, 1654, 1456, 1367, 1193, 1024, 699. The 1H-NMR (CDCl3): δ 7.2 (s, 5H), 4.4(m,4H), 4.2 (q,2H), 3.6-1.3 (m, 18H), 1.28(d+t,6H). CI Mass (m/z): 429.6(M-H).

### EXAMPLE 3

### Preparation of N-[(S)-1-(ethoxycarbonyl)-3-phenylpropyl]-L-alanylchloride Hydrochloride (X)

Into a 4 necked round bottom flask, methylene chloride (700 ml) and phosphorous pentachloride (1.55 eq.) were added and stirred for about 90 minutes at a temperature ranging from about 20° C. to about 25° C. The reaction mixture was then cooled to a temperature of about −10° C. and N-[(S)-1-(ethoxycarbonyl)-3-phenylpropyl]-L-alanine (100 g, 0.361 mole=1 equivalent) was added. The reaction mixture was maintained for about 5 hours at a temperature ranging from about −5° C. to about 0° C. Cyclohexane (2.0 L) was slowly added while maintaining a temperature below about −5° C. The reaction mixture was maintained for about 1 hour at a temperature ranging from about −5° C. to about 0° C. The solid was filtered under a nitrogen atmosphere. The material was dried at a temperature ranging from about 40° C. to about 45° C. under vacuum. Dry wt. 120 g.

### EXAMPLE 4

### Preparation of Trandolapril

Into a 4 necked round bottom flask, methylene chloride (2000 ml) and N-[(S)-1-(ethoxycarbonyl)-3-phenylpropyl]-L-alanyl chloride hydrochloride (120 g) were added and cooled to a temperature of about −5° C. Imidazole (400 g) was added to the reaction mixture and maintained for about 1 hour at a temperature below about 0° C. (2S,3aR,7aS)-perhydroindole-2-carboxylic acid (100 g, 0.594 mole=1 equivalent) was added slowly over about 45 minutes. The reaction mass was then stirred for about 2 hours at temperature of about −5° C. to about 0° C. The temperature was then raised to about 20° C. to about 25° C. and maintained for about 2 hours. Then a mixture of acetic acid (2 L) in water (1 L) was added and the temperature was maintained below about 5° C. and stirred for about 30 minutes. The methylene chloride layer was separated off. The methylene chloride layer was then washed with saturated brine solution (200 ml) and dried over sodium sulfate (10 g). Methylene chloride was then distilled off completely and isopropyl alcohol (300 ml) was added. The reaction mixture was heated to temperature ranging from about 65° C. to about 70° C. to get a clear solution. The reaction mass was then cooled very slowly to a temperature of about 25° C. over 2 hours and then further cooled to a temperature ranging from about 5° C. to about 10° C. and filtered. Material was then dried under vacuum at a temperature of about 40° C. (Weight: 130.0 g, purity: >99.5% by HPLC).

### EXAMPLE 5

### Preparation of Trandolapril

Into a 4 necked round bottom flask, methylene chloride (50 ml) and (2S,3aR,7aS)-perhydroindole-2-carboxylic acid (II) (5 g, 0.03 mole=1 equivalent) and cooled to a temperature ranging from about −5° C. to about 0° C. Triethylamine (1.2 eq.) was added and the reaction mixture was maintained at a temperature ranging from about −5° C. to about 0° C. over a period of about 30 minutes. The reaction mixture was stirred for about 15 minutes. Then trimethyl silyl chloride (1.2 eq.) in methylene chloride (10 ml) was added drop wise while maintaining a temperature ranging from about −5° C. to about 0° C. The reaction mass was then stirred for about 2 hours at a temperature ranging from about −5° C to about 0° C. to get the trimethyl silyl ester of (2S,3aR,7aS)-perhydroindole-2-carboxylic acid. Imidazole (4.0 eq.) was added at a temperature ranging from about −5° C. to about 0° C. N-[(S)-1-(ethoxycarbonyl)-3-phenylpropyl]-L-alanylchloride hydrochloride (1.2 eq.) was added at a temperature ranging from about −5° C. to about 0° C. over about 30 minutes. The reaction mixture was stirred for about 1 hour. The temperature was increased to about 25° C. to about 30° C. The reaction mixture was stirred for about 1 hour. Methylene chloride was recovered and the reaction mass quenched with acetic acid (2 eq.) in water (50 ml) at a temperature below about 10° C. The reaction mixture was stirred for about 15 minutes at a temperature ranging from about 25° C. to about 30° C. The mass was extracted twice with methylene chloride and the methylene chloride layer was charcoalized over charcoal and sodium sulfate. Methylene chloride was then distilled off completely and isopropyl alcohol (15 ml) was added and heated to a temperature ranging from about 65° C. to about 70° C. to get a clear solution. The reaction mass was then cooled very slowly to about 25° C. over about 2 hours and then further cooled to a temperature ranging from about 5° C. to about 10° C. and filtered. The material was then dried under vacuum at a temperature of about 40° C. (Weight: 7.0 g, purity: >99.5% by HPLC).

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

What is claimed is:

1. A process for the preparation of an intermediate of trandolapril, (2S,3aR,7aS)-perhydroindole-2-carboxylic acid of Formula II: 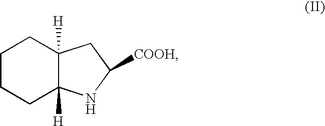
the process comprising:
(a) esterifying (3aR,7aS)-perhydroindole-2-carboxylic acid of Formula III: 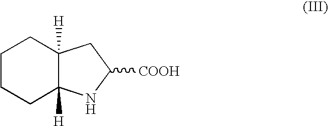
with an alcohol of the formula ROH wherein R is an aliphatic, aryl or aralkyl group and a suitable acid to provide an acid addition salt of the compound of Formula IV: 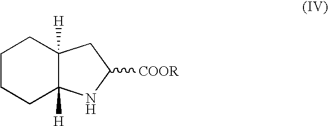
wherein R has the aforestated meanings;
(b) reacting the acid addition salt of the compound of Formula IV with a first base to provide a compound of Formula V: 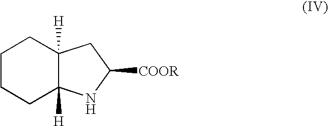
(c) reacting the compound of Formula IV with dibenzoyl-L-tartaric acid or di-p-toluoyl-L-tartaric acid and at least one alcohol of the formula R¹OH wherein R¹ is an alkyl group containing from 1 to about 12 carbon atoms, and adding a second base to precipitate a compound of Formula VI: 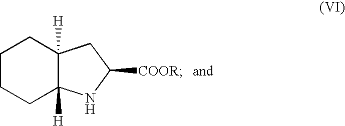
(d) hydrolyzing the compound of Formula VI to provide the compound of Formula II.

2. The process of claim 1, wherein the alcohol of the formula ROH is selected from the group consisting of a linear or branched C₁-C₃₀ aliphatic alcohol, C₆-C₃₀ aryl alcohol, C₆-C₃₀ araalkyl alcohol and mixtures thereof.

3. The process of claim 1, wherein the alcohol of the formula ROH is selected from the group consisting of methyl alcohol, ethyl alcohol, propyl alcohol, isopropyl alcohol, butyl alcohol, benzyl alcohol and mixtures thereof

4. The process of claim 1, wherein the suitable acid is a sulfonic acid.

5. The process of claim 4, wherein the sulfonic acid is p-toluenesulfonic acid.

6. The process of claim 1, wherein the suitable acid is a hydrochloric acid.

7. The process of claim 1, wherein step (a) is carried out in a solvent.

8. The process of claim 7, wherein the solvent is selected from the group consisting of an aromatic hydrocarbon, aliphatic hydrocarbons, halogenated aliphatic hydrocarbon, ether, nitrile and mixtures thereof.

9. The process of claim 7, wherein the solvent is selected from the group consisting n-hexane, benzene, toluene, o-, m-, and p-xylene (single or mixed isomers), ethylbenzene and mixtures thereof.

10. The process of claim 1, wherein the first base is an amine selected from the group consisting of a primary amine, secondary amine, tertiary amine, aliphatic amine, aromatic amine and mixtures thereof.

11. The process of claim 1, wherein the first base is one or more tertiary amines.

12. The process of claim 1, wherein the first base is selected from the group consisting of triethylamine, ammonia and mixtures thereof.

13. The process of claim 1, wherein the lower aliphatic alcohol of step (c) is selected from the group consisting of methanol, ethanol, butanol, propanol, isopropyl alcohol and mixtures thereof.

14. The process of claim 1, wherein in step (c) the compound of Formula IV is reacted with dibenzoyl-L-tartaric acid.

15. The process of claim 1, comprising:
(a) esterifying (3aR,7aS)-perhydroindole-2-carboxylic acid of Formula III: 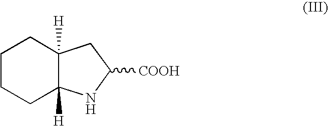
with benzyl alcohol and a suitable acid selected from the group consisting of p-toluenesulfonic acid and hydrochloric acid to provide an acid addition salt thereof;
(b) reacting the acid addition salt with a trialkylamine:
(c) reacting the product of step (b) with dibenzoyl-L-tartaric acid and at least one lower aliphatic alcohol of the formula R¹OH wherein R¹ is an alkyl group containing from 1 to about 6 carbon atoms, and adding a second base to precipitate a compound of Formula VI; and
(d) hydrolyzing the compound of Formula VI to provide the compound of Formula II.

16. The process of claim 1, and thereafter converting the compound of Formula II to trandolapril or a pharmaceutically acceptable salt thereof.

17. The process of claim 15, and thereafter converting the compound of Formula II to trandolapril or a pharmaceutically acceptable salt thereof.

18. A process for the preparation of trandolapril comprising:
(a) reacting N-[(S)-1-(ethoxycarbonyl)-3-phenylpropyl]-L-alanine and N,N′-carbonyldiimidazole in the presence of a solvent to form an N-carboxyanhydride; and
(b) reacting the N-carboxyanhydride with (2S,3aR,7aS)-perhydroindole-2-carboxylic acid to provide trandolapril.

19. The process of claim 18, wherein the solvent is selected from the group consisting of a halogenated hydrocarbon solvent, ether solvent, amide solvent and mixtures thereof.

20. The process of claim 18, wherein the solvent is one or more halogenated hydrocarbon solvents.

21. The process of claim 20, wherein the halogenated hydrocarbon solvent is selected from the group consisting of dichloromethane, dichloroethane and mixtures thereof.

22. The process of claim 18, wherein the solvent is selected from the group consisting of dichloromethane, dichloroethane, tetrahydrofuran, dimethylformamide and mixtures thereof.

23. A process for preparing trandolapril comprising reacting a N-[(S)-1-carbethoxybutyl]-(S)-alanyl halide of Formula X:
Me 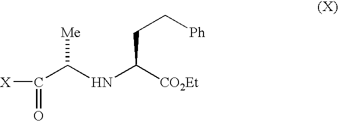
wherein X is a halide and Ph is phenyl with (2S,3aR,7aS)-perhydroindole-2-carboxylic acid of Formula II: 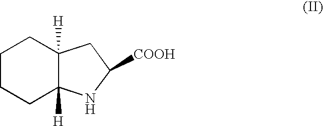
in the presence of a base to provide trandolapril.

24. The process of claim 23, wherein the base is an organic base selected from the group consisting of a dialkylamine, trialkylamine, heterocyclic amine and mixtures thereof.

25. The process of claim 23, wherein the base is selected from the group consisting of imidazole, triethyl amine, diethylamine, pyridine, n-methyl morpholine and mixtures thereof.

26. The process of claim 23, wherein the reaction takes place in one or more organic solvents.

27. The process of claim 26, wherein the one or more organic solvents is an anhydrous solvent.

28. The process of claim 27, wherein the one or more anhydrous solvents is selected from the group consisting of a chlorinated hydrocarbon, aromatic hydrocarbon, aliphatic hydrocarbon and mixtures thereof.

29. The process of claim 26, wherein the organic solvent is selected from the group consisting of dichloromethane, dichloroethane and mixtures thereof.

30. The process of claim 28, wherein the organic solvent is selected from the group consisting of benzene, toluene, and mixtures thereof.

31. The process of claim 28, wherein the organic solvent is selected from the group consisting of hexane, heptane, cyclopentane, cyclohexane and mixtures thereof.

32. The process of claim 23, wherein the N-[(S)-1-carbethoxybutyl]-(S)-alanyl halide of Formula X is formed by reacting N-[(S)-1-(ethoxycarbonyl)-3-phenylpropyl]-L-alanine with a halogenating agent in a suitable anhydrous solvent and in the presence or absence of an inert gas.

33. The process of claim 23, wherein the trandolapril is thereafter converted to a pharmaceutically acceptable salt thereof.

34. The process of claim 23, further comprising the step of adjusting the pH to about 4 to about 5.

35. The process of claim 23, further comprising the step of purifying trandolapril.

36. Trandolapril prepared in accordance with the process of claim 23 having a purity of greater than about 95%.

37. A process for preparing trandolapril comprising reacting (2S,3aR,7aS)-perhydroindole-2-carboxylic acid of Formula II: 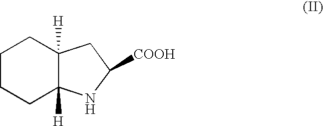
with a trialkyl or aryl silyl halide in the presence of a first base and in situ reacted with a N-[(S)-1-carbethoxybutyl]-(S)-alanyl halide of Formula X: 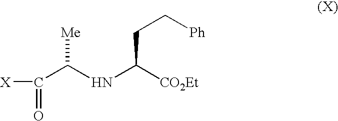
wherein X is a halide and Ph is phenyl in an organic solvent and in the presence of a second base followed by treatment with a suitable acid to obtain trandolapril.

38. The process of claim 37, wherein the second base is an organic base selected from the group consisting of a dialkylamine, trialkylamine, heterocyclic amine and mixtures thereof.

39. The process of claim 37, wherein the second base is selected from the group consisting of imidazole, triethylamine, diethylamine, pyridine, n-methyl morpholine and mixtures thereof.
